SEQUENCE LISTING (8 sequences)

SEQ ID NO: 1 - protein, 5 aa, Artificial
  Description of artificial sequence; amino acid sequence of AM-14_H_CDR1
RYGIS

SEQ ID NO: 2 - protein, 17 aa, Artificial
  Description of artificial sequence; amino acid sequence of AM-14_H_CDR2
WISTYSGNTNYAQKLQG

SEQ ID NO: 3 - protein, 7 aa, Artificial
  Description of artificial sequence; amino acid sequence of AM-14_H_CDR3
RQLYFDY

SEQ ID NO: 4 - protein, 11 aa, Artificial
  Description of artificial sequence; amino acid sequence of AM-14_L_CDR1
RASQSVSSNLA

SEQ ID NO: 5 - protein, 7 aa, Artificial
  Description of artificial sequence; amino acid sequence of AM-14_L_CDR2
DASTRAT

SEQ ID NO: 6 - protein, 9 aa, Artificial
  Description of artificial sequence; amino acid sequence of AM-14_L_CDR3
QQYDNWPLT

SEQ ID NO: 7 - protein, 116 aa, Artificial
  Description of artificial sequence; amino acid sequence of AM-14_VH
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 8 - protein, 107 aa, Artificial
  Description of artificial sequence; amino acid sequence of AM-14_VL
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

(19) United States
(12) Patent Application Publication
(10) Pub. No.: US 2017/0298136 A1
(43) Pub. Date: Oct. 19, 2017

(54) METHOD FOR TREATING PSORIASIS PATIENT WHICH RECEIVED ANTI-TNF-ALPHA ANTIBODY THERAPY

(71) Applicant: KIRIN-AMGEN, INC., Thousand Oaks, CA (US)

(72) Inventors: Hidemi NAKAGAWA, Tokyo (JP); Hiroki MATSUDO, Tokyo (JP)

(21) Appl. No.: 15/513,132

(22) PCT Filed: Aug. 26, 2015
(86) PCT No.: PCT/JP2015/004306

§ 371 Date: Mar. 21, 2017

Related U.S. Application Data
(60) Provisional application No. 62/041,862, filed on Aug. 26, 2014.

(51) Int. Cl. C07K 16/28; A61K 39/00

(52) U.S. Cl. CPC C07K 16/2866; C07K 2317/76; A61K 2039/505; A61K 2039/545; C07K 2317/21

(57) ABSTRACT

The present invention relates to a therapeutic agent for pustular psoriasis or psoriatic erythroderma that is administered to a psoriasis patient that has been administered with an anti-TNF-alpha antibody, comprising an IL-17RA antagonist as an active ingredient; and to a therapeutic agent for psoriasis that is administered to a psoriasis patient that cannot be treated with an anti-TNF-alpha antibody, comprising an IL-17RA antagonist as an active ingredient. In addition, the present invention also relates to a method for the treatment of pustular psoriasis or psoriatic erythroderma, comprising administering an IL-17RA antagonist to a psoriasis patient that has been administered with an anti-TNF-alpha antibody; and to a method for the treatment of psoriasis, comprising administering an IL-17RA antagonist to a psoriasis patient that cannot be treated with an anti-TNF-alpha antibody.

### TECHNICAL FIELD

The present invention relates to a therapeutic agent for pustular psoriasis or psoriatic erythroderma that is administered to a psoriasis patient that has been administered with an anti-TNF-alpha antibody, comprising an IL-17RA antagonist as an active ingredient; and to a therapeutic agent for psoriasis that is administered to a psoriasis patient that cannot be treated with an anti-TNF-alpha antibody, comprising an IL-17RA antagonist as an active ingredient. The present invention also relates to a method for the treatment of pustular psoriasis or psoriatic erythroderma, comprising administering an IL-17RA antagonist to a psoriasis patient that has been administered with an anti-TNF-alpha antibody; and to a method for the treatment of psoriasis, comprising administering an IL-17RA antagonist to a psoriasis patient that cannot be treated with an anti-TNF-alpha antibody.

### BACKGROUND ART

Overproduction of cytokines contributes to the development of a number of inflammatory autoimmune diseases including psoriasis, psoriatic arthritis, and asthma. The interleukin (IL)-17 family of cytokines is comprised of 6 family members with the following nomenclature: IL-17A, IL-17B, IL-17C, IL-17D, IL-25 (also known as IL-17E), and IL-17F. Interleukin-17A, IL-17F, and IL-17A/F are hallmark proinflammatory cytokines produced by T helper cells producing IL-17 (Th17) cells and innate immune cells that have been shown to contribute to an inflammatory response in models of autoimmune disorders (NPLs 1-6).

Interleukin-17A, IL-17F, and IL-17A/F have pleiotropic activities, including the induction of proinflammatory mediators from epithelial cells, endothelial cells and fibroblasts that promote tissue inflammation and destruction; the proliferation, maturation, and chemotaxis of neutrophils; and the maturation of dendritic cells (NPLs 7, 8).

Interleukin-25 is produced by epithelial cells, T-helper 2 (Th2) cells, eosinophils, and basophils and is more often associated with Th2-type inflammatory pathologies such as asthma (NPLs 9-11). Interleukin-17C is also produced by epithelial cells and the biological activities of this cytokine are being explored but appear to be similar to the activities of IL-17A and IL-17F (NPL 12).

Interleukin-17RA is a type I transmembrane receptor that is found on a wide variety of cell types including but not limited to fibroblasts, epithelial cells, and monocytes (NPL 13). Interleukin-17A, IL-17F, IL-17A/F, IL-17C, and IL-25 stimulate cellular responses by binding to IL-17RA. Interleukin-17A, IL-17F, and IL-17A/F signal via a heteromeric IL-17RA/IL-17RC complex, IL-17C signals via a heteromeric IL 17RA/IL-17RE complex, and IL-25 signals via a heteromeric IL-17RA/IL-17RB complex (NPLs 12, 14-17). Interleukin-17RA blockade represents a novel mechanism to inhibit the inflammation and clinical symptoms associated with autoimmune and inflammatory diseases including but not limited to psoriasis, psoriatic arthritis, and asthma.

AM-14 is a human CHO cell-derived IgG2 monoclonal antibody that binds to human IL-17RA and blocks the biological activities of IL-17A, IL-17F, IL-17A/F heterodimer, and IL-25 involved in the various autoimmune disorder and inflammatory disorder (NPLs 18-20).

### CITATION LIST

### Non Patent Literature

NPL 1: Nat Rev Immunol. 2010; 10:479-490
NPL 2: Cell. 2010; 140:845-858
NPL 3: Nature Rev Immunol.2009; 9(8):556-567
NPL 4: Immunity. 2008; 28(4):454-67
NPL 5: J Exp Med. 2005; 201(2):233-240
NPL 6: Nat Immunol. 2005; 6(11):1133-1141
NPL 7: Immunity.2004; 21:467-476
NPL 8: J Immunol. 1999; 162(1):577-584
NPL 9: Immunological Rev. 2008; 226:172-190
NPL 10: J Exp Med. 2007; 204(8):1837-1847
NPL 11: Immunity. 2001; 15:985-995
NPL 12: Nat Immunol.2011; 12(12):1159-1166
NPL 13: Cytokine. 1997; 9(11):794-800
NPL 14: Nat Immunol. 2011; 12(12):1151-1158
NPL 15: J Immunol. 2008; 181(6):4299-4310
NPL 16: J Immunol. 2008; 181(4):2799-2805
NPL 17: J Immunol. 2006; 177(1):36-39
NPL 18: Immunity. 2008; 28(4):454-67
NPL 19: J. Allergy. Clin. Immunol. 2007; 120(6):1324-31
NPL 20: J Immunol. 2005; 175(1):404-12

### SUMMARY OF INVENTION

### Technical Problem

It is an object of the present invention to provide a therapeutic agent for pustular psoriasis or psoriatic erythroderma that is administered to a psoriasis patient that has been administered with an anti-TNF-alpha antibody, comprising an IL-17RA antagonist as an active ingredient; and a therapeutic agent for psoriasis that is administered to a psoriasis patient that cannot be treated with an anti-TNF-alpha antibody, comprising an IL-17RA antagonist as an active ingredient. The present invention is also intended to provide a method for the treatment of pustular psoriasis or psoriatic erythroderma, comprising administering an IL-17RA antagonist to a psoriasis patient that has been administered with an anti-TNF-alpha antibody, and a method for the treatment of psoriasis, comprising administering an IL-17RA antagonist to a psoriasis patient that cannot be treated with an anti-TNF-alpha antibody.

### Solution to Problem

The present invention relates to the following (1) to (34).

(1) A therapeutic agent for pustular psoriasis or psoriatic erythroderma that is administered to a psoriasis patient that has been administered with an anti-TNF-alpha antibody, comprising an IL-17RA antagonist as an active ingredient.

(2) The therapeutic agent described in (1), wherein the anti-TNF-alpha antibody is at least one antibody selected from adalimumab, infliximab, certolizumab pegol, certolizumab, and golimumab.

(3) The therapeutic agent described in (1) or (2), wherein the IL-17RA antagonist is an anti-IL-17RA antibody or an antibody fragment thereof.

(4) The therapeutic agent described in (3), wherein the anti-IL-17RA antibody is selected from the following a) and b):

a) a monoclonal antibody in which a complementarity determining region (hereinafter, referred to as CDR)1, CDR2, and CDR3 of a heavy chain variable region (hereinafter, referred to as VH) of the antibody comprise the amino acid sequences shown in SEQ ID NOs:1, 2, and 3, respectively, and CDR1, CDR2, and CDR3 of a light chain variable region (hereinafter, referred to as VL) of the antibody comprise the amino acid sequences shown in SEQ ID NOs:4, 5, and 6, respectively; and

b) a monoclonal antibody in which VH of the antibody comprises the amino acid sequence shown in SEQ ID NO:7, and VL of the antibody comprises the amino acid sequence shown in SEQ ID NO:8.

(5) The therapeutic agent described in any one of (1) to (4), wherein the clinical global impression (hereinafter, also referred to as CGI) of a patient after administration of the therapeutic agent becomes 2 or 1.

(6) The therapeutic agent described in any one of (1) to (5), wherein psoriasis area and severity index (hereinafter, referred to as PASI) score of a patient after administration of the therapeutic agent is lower than that before administration of the therapeutic agent.

(7) A therapeutic agent for psoriasis that is administered to a psoriasis patient that cannot be treated with an anti-TNF-alpha antibody, comprising an IL-17RA antagonist as an active ingredient.

(8) The therapeutic agent described in (7), wherein the patient that cannot be treated with the anti-TNF-alpha antibody is a patient that does not respond to the antiTNF-alpha antibody or that is of insufficient tolerability to the anti-TNF-alpha antibody.

(9) The therapeutic agent described in (7) or (8), wherein the anti-TNF-alpha antibody is at least one selected from adalimumab, infliximab, certolizumab pegol, certolizumab, and golimumab.

(10) The therapeutic agent described in any one of (7) to (9), wherein the IL-17RA antagonist is an anti-IL-17RA antibody or an antibody fragment thereof.

(11) The therapeutic agent described in (10), wherein the anti-IL-17RA antibody is selected from the following a) and b):

a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and

b) a monoclonal antibody in which VH of the antibody comprises the amino acid sequence shown in SEQ ID NO:7, and VL of the antibody comprises the amino acid sequence shown in SEQ ID NO:8.

(12) The therapeutic agent described in any one of (7) to (11), wherein the psoriasis is at least one psoriasis selected from psoriasis vulgaris, psoriasis arthropica, pustular psoriasis, psoriatic erythroderma, and psoriasis guttata.

(13) The therapeutic agent described in any one of (7) to (12), wherein the CGI of a patient after administration of the therapeutic agent becomes 2 or 1.

(14) The therapeutic agent described in any one of (7) to (13), wherein the PASI score of a patient after administration of the therapeutic agent is lower than that before administration of the therapeutic agent.

(15) A method for the treatment of pustular psoriasis or psoriatic erythroderma, comprising administering an IL-17RA antagonist to a psoriasis patient that has been administered with an anti-TNF-alpha antibody.

(16) The method described in (15), wherein the anti-TNF-alpha antibody is at least one selected from adalimumab, infliximab, certolizumab pegol, certolizumab, and golimumab.

(17) The method described in (15) or (16), wherein the IL-17RA antagonist is an anti-IL-17RA antibody or an antibody fragment thereof.

(18) The method described in (17), wherein the anti-IL-17RA antibody is selected from the following a) and b):

a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and

b) a monoclonal antibody in which VH of the antibody comprises the amino acid sequence shown in SEQ ID NO:7, and VL of the antibody comprises the amino acid sequence shown in SEQ ID NO:8.

(19) The method described in any one of (15) to (18), wherein the CGI of the patient after the administration of the IL-17RA antagonist becomes 2 or 1.

(20) The method described in any one of (15) to (19), wherein the PASI score of a patient after the administration of the IL-17RA antagonist is lower than that before the administration of the antagonist.

(21) The method described in any one of (15) to (20), wherein the IL-17RA antagonist is administered in a dose of 140 mg or more.

(22) The method described in any one of (15) to (21), wherein the IL-17RA antagonist is administered in a dose of 140 mg or 210 mg.

(23) The method described in any one of (15) to (22), wherein the IL-17RA antagonist is administered in a dose of 140 mg or 210 mg on the 1^{st} day, the 1^{st} week and the 2^{nd} week, and thereafter once every two weeks.

(24) A method for the treatment of psoriasis, comprising administering an IL-17RA antagonist to a psoriasis patient that cannot be treated with an anti-TNF-alpha antibody.

(25) The method described in (24), wherein the patient that cannot be treated with the anti-TNF-alpha antibody is a patient that is ineffective to the anti-TNF-alpha antibody or that is of insufficient tolerability to the anti-TNF-alpha antibody.

(26) The method described in (24) or (25), wherein the anti-TNF-alpha antibody is at least one selected from adalimumab, infliximab, certolizumab pegol, certolizumab, and golimumab.

(27) The method described in any one of (24) to (26), wherein the IL-17RA antagonist is an anti-IL-17RA antibody or an antibody fragment thereof.

(28) The method described in (27), wherein the anti-IL-17RA antibody is selected from the following a) and b):

a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and

b) a monoclonal antibody in which VH of the antibody comprises the amino acid sequence shown in SEQ ID NO:7, and VL of the antibody comprises the amino acid sequence shown in SEQ ID NO:8.

(29) The method described in any one of (24) to (28), wherein the psoriasis is at least one selected from psoriasis vulgaris, psoriasis arthropica, pustular psoriasis, psoriatic erythroderma, and psoriasis guttata.

(30) The method described in any one of (24) to (29), wherein the CGI of the patient after the administration of the IL-17RA antagonist becomes 2 or 1.

(31) The method described in any one of (24) to (30), wherein the PASI score of a patient after the administration of the IL-17RA antagonist is lower than that before the administration of the antagonist.

(32) The method described in any one of (24) to (31), wherein the IL-17RA antagonist is administered in a dose of 140 mg or more.

(33) The method described in any one of (24) to (32), wherein the IL-17RA antagonist is administered in a dose of 140 mg or 210 mg.

(34) The method described in any one of (24) to (33), wherein the IL-17RA antagonist is administered in a dose of 140 mg or 210 mg on the 1^{st} day, the 1^{st} week and the 2^{nd} week, and thereafter once every two weeks.

### Advantageous Effects of Invention

The present invention can provide a therapeutic agent for pustular psoriasis or psoriatic erythroderma that is administered to a psoriasis patient that has been administered with an anti-TNF-alpha antibody, comprising an IL-17RA antagonist as an active ingredient; and a therapeutic agent for psoriasis that is administered to a psoriasis patient that cannot be treated with an anti-TNF-alpha antibody, comprising an IL-17RA antagonist as an active ingredient. The present invention can also provide a method for the treatment of pustular psoriasis or psoriatic erythroderma, comprising administering an IL-17RA antagonist to a psoriasis patient that has been administered with an anti-TNF-alpha antibody; and a method for the treatment of psoriasis, comprising administering an IL-17RA antagonist to a psoriasis patient that cannot be treated with an anti-TNF-alpha antibody.

### BRIEF DESCRIPTION OF DRAWINGS

FIGS. 1(A) to 1(C) show the therapeutic effect of AM-14 for pustular psoriasis patients that have been administered with anti-TNF-alpha antibodies. The vertical axis represents clinical global impression, and the horizontal axis represents a period after the first AM-14 administration (week). (A) to (C) are results for subjects A to C, respectively.

FIGS. 2(D) and 2(E) show the therapeutic effect of AM-14 for pustular psoriasis patients that have been administered with anti-TNF-alpha antibodies. The vertical axis represents clinical global impression, and the horizontal axis represents a period after the first AM-14 administration (week). (D) and (E) are results for subjects D and E, respectively.

FIG. 3 shows the therapeutic effect of AM-14 for psoriatic erythroderma patients that have been administered with anti-TNF-alpha antibodies. The vertical axis represents PASI score, and the horizontal axis represents a period (week) after the first AM-14 administration. Week 0 of administration is also denoted as the 1^{st} day of administration. The results for subjects F, G, and H are plotted in solid lines with the black circle, plus, and minus symbols, respectively.

### DESCRIPTION OF EMBODIMENTS

The present invention relates to a therapeutic agent for pustular psoriasis or psoriatic erythroderma that is administered to a psoriasis patient that has been administered with an anti-tumor necrosis factor-alpha (hereinafter, referred to as “TNF-alpha”) antibody, comprising an IL-17RA antagonist as an active ingredient; and to a therapeutic agent for psoriasis that is administered to a psoriasis patient that cannot be treated with an anti-TNF-alpha, comprising an IL-17RA antagonist as an active ingredient (hereinafter, the therapeutic agents also will be collectively referred to as “therapeutic agent of the present invention”).

The present invention also relates to a method for the treatment of pustular psoriasis or psoriatic erythroderma, comprising administering an IL-17RA antagonist to a psoriasis patient that has been administered with an anti-TNF-alpha antibody; and to a method for the treatment of psoriasis, comprising administering an IL-17RA antagonist to a psoriasis patient that cannot be treated with an anti-TNF-alpha antibody (hereinafter, the therapeutic methods also will be collectively referred to as “therapeutic method of the present invention”).

The anti-TNF-alpha antibody is not particularly limited in the present invention, as long as it is an antibody against TNF-alpha. The anti-TNF-alpha antibody is preferably an antibody against human TNF-alpha. Specific examples of the anti-human TNF-alpha antibody include infliximab, adalimumab, certolizumab pegol, certolizumab, golimumab, and the like.

Psoriasis is a chronic skin disease, and in clinical practice, psoriasis is classified into five types, that is, psoriasis vulgaris, psoriasis arthropica, pustular psoriasis, psoriatic erythroderma, and psoriasis guttata, according to the symptom and the expression site. Psoriasis vulgaris is characterized by skin erythema with thickening of the skin, clear boundaries (plaque), and scales. Psoriasis arthropica is a chronic inflammatory disease of disordering joints, tendon sheaths, tendon attachment portions, and an axial skeleton, in addition to the skin or the like. Pustular psoriasis is characterized by forming skin rash called a pustule on erythema. Psoriatic erythroderma is characterized by skin rash over the whole body, and being accompanied with diffuse blush or scales. Psoriasis guttata is characterized by existence of small size of psoriasis rash having a diameter of 1 cm on trunk, and legs and arms.

Examples of the psoriasis concerned with the present invention include psoriasis vulgaris, psoriasis arthropica (also called psoriatic arthropathy), pustular psoriasis, psoriatic erythroderma, psoriasis guttata, and the like. Preferred are psoriasis vulgaris, psoriasis arthropica, pustular psoriasis, and psoriatic erythroderma. More preferred are moderate to severe psoriasis vulgaris and psoriasis arthropica, and pustular psoriasis and psoriatic erythroderma. Further preferred are pustular psoriasis and psoriatic erythroderma. The psoriasis concerned with the present invention includes one that is complicated by plural types of psoriasis described above, and also includes nail psoriasis.

The psoriasis may be of a form involving plaque psoriasis, and is preferably a form involving moderate to severe plaque psoriasis. The psoriasis may be localized or may affect the whole body. Specific examples of localized psoriasis include scalp psoriasis and the like. The psoriasis involving plaque psoriasis mainly refers to psoriasis vulgaris involving plaque psoriasis, and also includes psoriasis arthropica involving joint symptoms in addition to plaque psoriasis.

The pustular psoriasis may be generalized type pustular psoriasis in which pustule appears extensively or localized type pustular psoriasis in which pustule appears in small range, and is preferably generalized type pustular psoriasis. Specific examples of generalized type pustular psoriasis include acute generalized pustular psoriasis, generalized pustular psoriasis, annular and circulate pustular psoriasis, impetigo herpetiformis, afebrile impetigo herpetiformis, juvenile and infantile pustular psoriasis, and the like. Specific examples of localized type pustular psoriasis include palmoplantar pustulosis, acrodermatitis continua, transient localized pustulization of psoriasis vulgaris, and the like.

The therapeutic agent of the present invention means a drug that can lower the degree of severity of pustular psoriasis or psoriatic erythroderma in a psoriasis patient that has been administered with an anti-TNF-alpha antibody. The therapeutic agent of the present invention also means a therapeutic agent that can lower the degree of severity of psoriasis in a psoriasis patient that cannot be treated with an anti-TNF-alpha antibody.

The therapeutic method of the present invention means a method that can lower the degree of severity of pustular psoriasis or psoriatic erythroderma in a psoriasis patient that has been administered with an anti-TNF-alpha antibody. The method of the present invention also means a therapeutic method that can lower the degree of severity of psoriasis in a psoriasis patient that cannot be treated with an anti-TNF-alpha antibody.

In the present invention, with regard to the term “that has been administered with an anti-TNF-alpha antibody”, it is not particularly limited as to when the anti-TNF-alpha antibody is administered, as long as it is administered to a patient before the therapeutic agent of the present invention or the IL-17RA antagonist in the therapeutic method of the present invention is first administered to the patient. After the antiTNF-alpha antibody is administered, another drug may be administered before the therapeutic agent of the present invention or the IL-17RA antagonist is first administered.

Examples of the psoriasis patient that cannot be treated with an anti-TNF-alpha antibody include a patient that is ineffective to the anti-TNF-alpha antibody (primary failure or secondary failure) or that is of insufficient tolerability when the antiTNF-alpha antibody is administered to a psoriasis patient.

As used herein, “primary failure” means that the drug efficacy is insufficient from the first administration, and “secondary failure” means that the efficacy of an administered drug attenuates over the course of the continuous administration. In addition, “insufficient tolerability” means that severe side effects to a degree at which a patient cannot tolerate occurs by the administered drug.

Any improvement of psoriasis symptoms may be determined by using known methods. For example, improvement can be determined by a reduction in the scores of various indices after the administration of the therapeutic agent of the present invention or the IL-17RA antagonist in the therapeutic method of the present invention relative to before the administration, specifically by a reduction in the score of Psoriasis Area and Severity Index (hereinafter, referred to as “PASI”), Nail Psoriasis Severity Index (hereinafter, referred to as “NAPSI”), Psoriasis Scalp Severity Index (hereinafter, referred to as “PSSI”), or static Physician Global Assessments (hereinafter, referred to as “sPGA”); the improvement of Quality of life (hereinafter, referred to as QOL) of a psoriasis patient; and the like.

PASI indicates a degree of severity of skin symptoms of psoriasis, and it can be measured according to the method described in [http://www.kyowa-kirin.co.jp/kayumi/kansen/jinjo_08.html]. NAPSI indicates a degree of severity of symptoms of nail psoriasis, and it can be measured according to the method described in Rich et al. (J. Am. Acad. Dermatol. vol. 49, number 2, p. 206-212 (2003)). PSSI indicates a degree of severity of scalp psoriasis, and it can be measured according to the method described in Leonardi et al. (J. Engl. J. Med. 2012; 366: 1190-9). sPGA indicates a degree of severity of skin symptoms comprehensively evaluated by a physician, and it can be measured according to the method described in H. N. Viswanathan et al. (Journal of Dermatological Treatment Posted online on Jul. 31, 2014. (doi:10.3109/09546634.2014.943687)). QOL uses DLQI as an indicator, and it can be measured according to the method described in H. N. Viswanathan et al. (Journal of Dermatological Treatment Posted online on Jul. 31, 2014. (doi:10.3109/09546634.2014.943687)).

Effects of the therapeutic agent of the present invention or the IL-17RA antagonist in the therapeutic method of the present invention is evaluated by the clinical global impression (hereinafter, referred to as CGI) which determines the change in skin symptoms of psoriasis from the first administration of the antagonist as one of four stages [1: remission, 2: improvement, 3: unchanged, and 4: aggravation], and as a result, the effects can also be confirmed from the fact that CGI becomes 1 (remission) or 2 (improvement).

Improvement of pustular psoriasis symptoms can also be confirmed from the fact that the degree score is reduced after the administration of the therapeutic agent of the present invention or the antagonist relative to before the administration, or the like, in addition to the method described above. The degree score is a score obtained by scoring the evaluation of skin symptoms (erythema, pustule, edema) of a patient and laboratory findings (fever, leukocyte count, serum CRP level, and serum albumin level) by being accompanied with systemic inflammation, and the degree score can be calculated based on the criteria of the degree of severity of pustular psoriasis described in “Clinical Practice Guideline 2010 for Pustular Psoriasis (generalized type): therapeutic strategy including incorporation of TNF-alpha inhibitor (Iwatsuki Keiji et al.)”.

Specifically, examples of an improvement of psoriatic erythroderma symptoms include a reduction of PASI score after the administration of the therapeutic agent or the antagonist relative to before the administration, and the like. More specifically, an improvement of psoriatic erythroderma symptoms means a 10%, 20%, 30%, 40%, 50%, 60%, 70%, 80%, 90%, or 100% reduction, preferably a 100% reduction of PASI score after the administration of the therapeutic agent relative to before the administration.

The term administration as used herein may refer to single administration or multiple administrations (hereinafter, also referred to as “continuous administration”). Further, when comparing the scores indicating the degrees of severity of psoriasis described above before and after administration, the score of the first administration and the score after single or multiple administrations can be compared, or the scores between suitable two points in continuous administration can be compared.

A dose of the therapeutic agent of the present invention and the IL-17RA antagonist in the method of the present invention is not particularly limited, and is desirably 70 mg or more, more desirably 140 mg or more, most desirably 210 mg or more. The dose can be suitably increased or decreased during the continuous administration of the therapeutic agent or the antagonist.

The dosing interval of the therapeutic agent and the antagonist is not particularly limited. Specifically, for example, the therapeutic agent and the antagonist may be administered on the 1^{st} day (hereinafter, also referred to as week 0), the 1^{st} week and the 2 nd week, and thereafter once every two weeks or once every four weeks. The dosing interval can also be appropriately extended, or can be shortened.

The dosing period of the therapeutic agent and the antagonist is not particularly limited. Specifically, for example, the therapeutic agent and the antagonist can be administered for 10, 20, 30, 40, or 50 weeks, or longer than 50 weeks after the first administration, and is preferably administered for longer than 50 weeks. The dosing period can include a rest period.

The IL-17RA in the present invention is not limited to a particular species, and is preferably human IL-17RA. The IL-17RA antagonist in the present invention can be any antagonist, as long as it inhibits the interaction between IL-17RA and a IL-17RA ligand (such as IL-17A, IL-17F, IL-17A/F, IL-25, or the like). Specifically, for example, the IL-17RA antagonist can be an antagonist having the activity to inhibit the binding of IL-17RA and the ligand, an antagonist having the activity to inhibit the IL-17RA activation initiated by the binding of the ligand, or an antagonist having the activity to inhibit the association between IL-17RA and a receptor forming a heterodimer.

The IL-17RA antagonist can have any form, including, for example, a small molecule, an antibody, an antibody fragment, an antigen-binding fragment, siRNA, an anti-sense oligomer, and the like. Preferably, the antagonist is an antibody, or an antibody fragment.

Specific examples of antibodies as IL-17RA antagonists include anti-IL-17RA antibody, anti-IL-17A antibody, anti-IL-17F antibody, anti-IL-17A/F antibody, and anti-IL-17E antibody. Preferred is anti-IL-17RA antibody.

The antibody used in the present invention can be any one of monoclonal antibody and polyclonal antibody, and preferably a monoclonal antibody which binds to a single epitope.

The antibody can be a monoclonal antibody produced from hybridomas or a genetically recombinant antibody produced by a gene recombination technique. Examples of the genetically recombinant antibody include a mouse antibody, a rat antibody, a human chimeric antibody (hereinafter, simply referred to as chimeric antibody), a humanized antibody [also called human complementarity determining region (CDR)-grafted antibody], and a human antibody; and in order to reduce immunogenicity in humans, a chimeric antibody, a humanized antibody, or a human antibody is preferably used.

Specifically, the monoclonal antibody of the present invention can be an antibody selected from the following (A) to (B):

(A) a monoclonal antibody in which CDR1, CDR2 and CDR3 of a heavy chain variable region (hereinafter, referred to as VH) of the antibody comprise the amino acid sequences shown in SEQ ID NOs:1, 2 and 3, respectively, and CDR1, CDR2 and CDR3 of a light chain variable region (hereinafter, referred to as VL) of the antibody comprise the amino acid sequences shown in SEQ ID NOs:4, 5 and 6, respectively; and

(B) a monoclonal antibody in which VH of the antibody comprises the amino acid sequence shown in SEQ ID NO:7, and VL of the antibody comprises the amino acid sequence shown in SEQ ID NO:8.

In the present invention, one embodiment of the monoclonal antibody in which CDR1, CDR2 and CDR3 of VH of the antibody comprise the amino acid sequences shown in SEQ ID NOs:1, 2 and 3, respectively, and CDR1, CDR2 and CDR3 of VL of the antibody comprise the amino acid sequences shown in SEQ ID NOs:4, 5 and 6, respectively or the monoclonal antibody in which VH of the antibody comprises the amino acid sequence shown in SEQ ID NO:7, and VL of the antibody comprises the amino acid sequence shown in SEQ ID NO:8 includes anti-human IL-17RA human monoclonal antibody AM_{H}14/AM_{L}14 (WO 2008/054603), and genetically recombinant anti-human IL-17RA human antibody AM-14 (also referred to as brodalumab), and the like.

In the present invention, the monoclonal antibody refers to an antibody that is secreted by an antibody-producing cell of a single clone, and recognizes only one epitope (antigenic determinant), and the amino acid sequence (primary structure) constituting the monoclonal antibody is uniform.

The epitope refers to a single amino acid sequence, a three-dimensional structure consisting of the amino acid sequence, an amino acid sequence bound with a modified residue such as a sugar chain, glycolipid, polysaccharide lipid, an amino group, a carboxyl group, phosphoric acid, sulfuric acid, and the like, and a three-dimensional structure consisting of said modified residue-bound amino acid sequence, which the monoclonal antibody recognizes for binding. The three-dimensional structure is a three-dimensional structure of naturally occurring proteins, which refers to a three-dimensional structure constituted with proteins that are expressed intracellularly or on a cell membrane.

In the present invention, the antibody molecule is also called immunoglobulin (hereinafter, referred to as “Ig”). Human antibodies are classified into isotypes of IgA1, IgA2, IgD, IgE, IgG1, IgG2, IgG3, IgG4 and IgM according to the difference between molecular structures. IgG1, IgG2, IgG3, and IgG4 which are relatively highly homologous to each other in terms of the amino acid sequence are also collectively called IgG.

In the present invention, the antibody molecule consists of polypeptides called a heavy chain (hereinafter, referred to as H-chain) and a light chain (hereinafter, referred to as L-chain).

Further, the H chain consists of VH and an H-chain constant region (also referred to as CH) from the N-terminal, and the L chain consists of VL and an L-chain constant region (also referred to as CL) from the N-terminal, respectively.

A chimeric antibody refers to an antibody which consists of VH and VL of an antibody derived from a non-human animal and CH and CL of a human antibody. The species of animal from which the variable region is derived is not particularly limited, as long as it is an animal that can be used for the preparation of hybridomas, such as mouse, rat, hamster, rabbit or the like.

A humanized antibody is an antibody which is obtained by grafting CDRs of VH and VL derived from a non-human animal antibody into appropriate positions of VH and VL of a human antibody (human CDR-grafted antibody).

A human antibody means an antibody naturally existing in the human body, and also includes an antibody obtained from a human antibody phage library or a human antibody-producing transgenic animal, which is prepared based on the recent advanced techniques in genetic engineering, cell engineering and developmental engineering.

In the present invention, the type of the antibody fragment is not particularly limited, and examples thereof can include Fab, Fab′, F(ab′)₂, scFv, diabody, dsFv, a peptide containing CDR and the like.

The therapeutic agent of the present invention can be one which includes only the IL-17RA antagonist as an active ingredient, however, it is desirable to provide as a pharmaceutical formulation which is usually mixed together with one or more pharmacologically acceptable carriers and is manufactured by any well-known method in a technical field of pharmaceutics. Specific examples of the pharmaceutical formulation include a pharmaceutical formulation which comprises 140 mg/mL of genetically recombinant anti-human IL-17RA human antibody AM-14 as an active ingredient and which is formulated with 10 mmol/L of L-glutamic acid, 3% (w/v) of L-proline and 0.010% (w/v) of polysorbate 20 as an additive agent, and the like. The pharmaceutical formulation also includes a pharmaceutical formulation of pH 4.8 which comprises 140 mg/mL of a genetically recombinant anti-human IL-17RA human antibody AM-14 and further comprises 30 mmol/L of L-glutamic acid, 2.4% (w/v) of L-proline, and 0.01% (w/v) of polysorbate 20 as an additive agent. The pharmaceutical formulation can be produced, for example, by the method described in WO2011/088120.

As to an administration route of the therapeutic agent of the present invention or the IL-17RA antagonist which is administered in the method of the present invention, it is preferable to use one which is most effective during treatment. Specific examples of the administration route include an oral administration or a parenteral administration such as intraoral, airway, intrarectal, subcutaneous, intramuscular or intravenous administration, and a subcutaneous or intravenous administration is preferable and a subcutaneous administration is more preferable. Examples of an administration form include spray, capsule, tablet, powder, granule, syrup, emulsion, suppository, injection, ointment, tape and the like and injection is preferable.

An administration device of the therapeutic agent or the IL-17RA antagonist of the present invention can be appropriately selected during treatment. Examples thereof include a prefilled syringe and an auto injector, however, the device is not limited to the examples.

### Example 1

Phase III Clinical Trial of Recombinant Anti-Human IL-17RA Human Antibody AM-14 in Japan

The outline of the phase III clinical trial (hereinafter, also referred to simply as “clinical trial”) of the recombinant anti-human IL-17RA human antibody AM-14 (hereinafter, “AM-14”) in Japan is presented in Table 1. Pustular psoriasis patients or psoriatic erythroderma patients that satisfy the conditions presented in Table 2 were selected as clinical trial subjects, and evaluated for the safety and efficacy or the like of AM-14 after chronic administration. Each subject was administered with 140 mg of AM-14 on the 1^{st} day, the 1^{st} week and the 2^{nd} week, and thereafter once every two weeks to the 50^{th} week. For subjects in which a sufficient effect was not obtained at the 4^{th} week, the dose was increased and 210 mg of AM-14 was administered once every two weeks from the 6^{th} week. The clinical trial ended at the 52^{th} week after the administration.

AM-14 had an antibody variable region amino acid sequences described in WO 2008/054603, and was prepared as a recombinant human antibody derived from a CHO cell by using an ordinary method.

TABLE 1

| | | No. Subjects |
|---|---|---|
| Indication, Design | Dose, Duration | Enrolled |
| Indication; generalized | Dose; 140 mg (dosage may | 12 patients (GPP), |
| pustular psoriasis | be increased to 210 mg in | 18 patients (PsE) |
| (GPP) or psoriatic | case of insufficient efficacy) | |
| erythroderma (PsE) | Duration; 50 weeks | |
| Design; Open-label, | | |
| Uncontrolled Study | | |

TABLE 2
Ages Eligible for Study: 18 Years and older
Genders Eligible for Study: Both
Accepts Healthy Volunteers: No
Criteria
1) Inclusion Criteria:
Subject has signed voluntarily the written informed consent form
to participate in this study.
Subject has been diagnosed as pustular psoriasis or
psoriatic erythroderma.
Subject has received at least one previous phototherapy
or systemic psoriasis therapy or has been a candidate to
receive phototherapy or systemic psoriasis therapy in the
opinion of the investigator.
2) Exclusion Criteria:
Subject with psoriatic erythroderma has involved Body surface area
(BSA) of lesion <80% at baseline.
Subject diagnosed with psoriasis guttata, medication-induced or
medication-exacerbated psoriasis.
Evidence of skin conditions at the time of the screening visit (eg, eczema)
that would interfere with evaluations of the effect of AM-14 on psoriasis.
Subject has any active Common Terminology Criteria for Adverse Events
(CTCAE) grade 2 or higher infection
Subject has a significant concurrent medical condition or laboratory
abnormalities, as defined in the study protocol.
Subject has used Ultra Violet B (UVB) therapy within 14 days of the
first dose or Ultra Violet A (UVA) (with or without psoralen) within
28 days of the first dose.
Subject has used etanercept, adalimumab, infliximab
or ustekinumab within 1 week, 2 weeks, 8 weeks or 12
weeks of the first dose, respectively.
Subject has stopped ustekinumab or other anti-Interleukin (IL)-23
biologics therapydue to lack of efficacy
Subject has used live vaccine within 3 months of the first dose
Subject has previously used an anti-IL-17 biologic therapy

### Example 2

Therapeutic Effect of AM-14 on Pustular Patients that was administered with an Anti-TNF-alpha Antibody

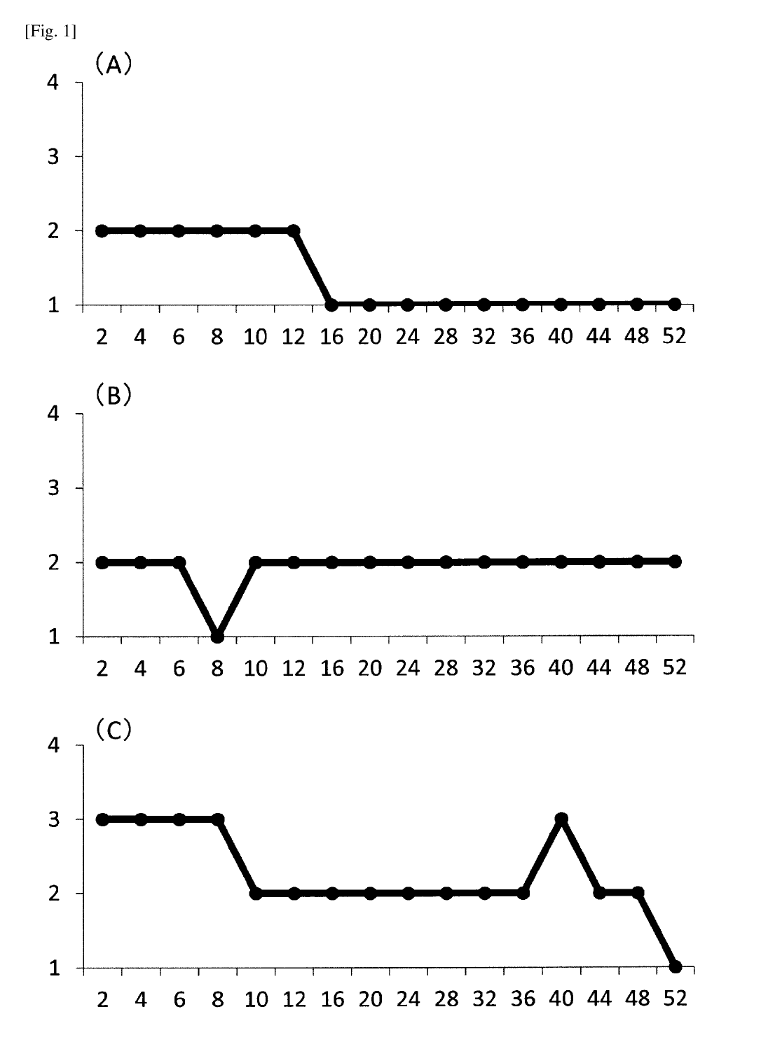

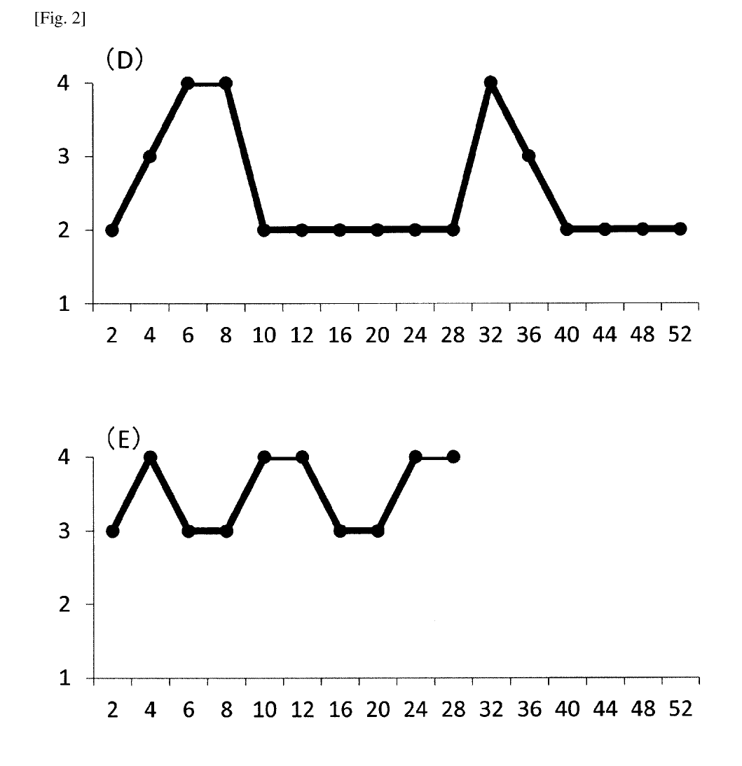

Five of the subjects participated in the clinical trial of Example 1 had been administered with the anti-TNF-alpha antibody. These subjects were measured for the clinical global impression (hereinafter, referred to as CGI) which represents the change in skin symptoms of psoriasis from the first administration of AM-14, over a time course. The obtained results are presented in FIGS. 1(A) to 1(C), 2(D) and 2(E). CGI was shown in scales of 1 to 4 representing remission, improvement, unchanged, and aggravation, respectively, as determined by physician.

As shown in FIGS. 1(A) to 1(C), 2(D) and 2(E), AM-14 were able to be administered for 50 weeks to four out of the five pustular patients that had been administered with the anti-TNF-alpha antibody, and the CGI at the 52^{th} week was 2 or less. As demonstrated above, AM-14 was shown to have a therapeutic effect for pustular patients that had been administered with the anti-TNF-alpha antibody.

### Example 3

Therapeutic Effect of AM-14 on Pustular Psoriasis Patients that could not be treated with an Anti-TNF-alpha Antibody

Table 3 presents histories of psoriasis therapeutic agent use in the five subjects examined in Example 2. Subjects B and D in Table 3 used the psoriasis therapeutic agents in the orders shown. The three subjects A, C, and E had been using infliximab until just before AM-14 administration. Infliximab and adalimumab are anti-human TNF-alpha antibodies (infliximab is a chimeric antibody, and adalimumab is a humanized antibody), and ustekinumab is an anti-human IL-12/23p40 humanized antibody. These psoriasis therapeutic agents are all approved in Japan. The reason for withdrawal of these drugs is given as “primary failure” when the drug efficacy was insufficient from the start of administration, and “secondary failure” when the drug efficacy attenuated over the course of continuous administration in Table 3. A case where the drug administration needed to be discontinued because of not efficacy but severe side effects to a degree at which a subject cannot tolerate was described as insufficient tolerability in Table 3. AM-14 was determined as being therapeutically effective when the CGI at the 52^{th} week was 2 or 1, and ineffective when the CGI score was other than 2 and 1.

TABLE 3
Histories of Using Psoriasis Therapeutic Agent

| | | Reason for withdrawal of | Therapeutic |
|---|---|---|---|
| | Psoriasis therapeutic | psoriasis therapeutic | effect |
| Subject | agent administered | agent administered | of AM-14 |
| A | Infliximab | Primary failure | Present |
| B | Infliximab | Secondary failure | Present |
| | Adalimumab | Secondary failure | |
| | Ustekinumab | Started a new treatment | |
| C | Infliximab | Secondary failure | Present |
| D | Infliximab | Insufficient tolerability | Present |
| | Ustekinumab | Stopped hospital visit | |
| | Adalimumab | Insufficient tolerability | |
| E | Infliximab | Secondary failure | Absent |
| | | | (withdrawn |
| | | | during trial) |

As shown in Table 3, the AM-14 administration was shown to have a therapeutic effect in three (subjects A, B, and C) of the four subjects (A, B, C, and E) that did not respond to infliximab or adalimumab (primary failure or secondary failure). As shown in the results of the subject D, it was found that AM-14 was able to be continuously administered for 50 weeks and shown therapeutic effect even for subjects that suffered from severe side effects by administration of infliximab and adalimumab and were determined to be insufficient tolerability to infliximab and adalimumab.

As demonstrated by these results, it was shown that, for a subject having pustular psoriasis that could not be treated with the anti-TNF-alpha antibody because the antiTNF-alpha antibody such as infliximab and adalimumab does not work, or causes severe side effects, AM-14 is able to be continuously administered over a long period of time and to improve psoriasis symptoms.

### Example 4

Therapeutic Effect of AM-14 on Psoriatic Erythroderma patients that have been administered with an Anti-TNF-alpha Antibody

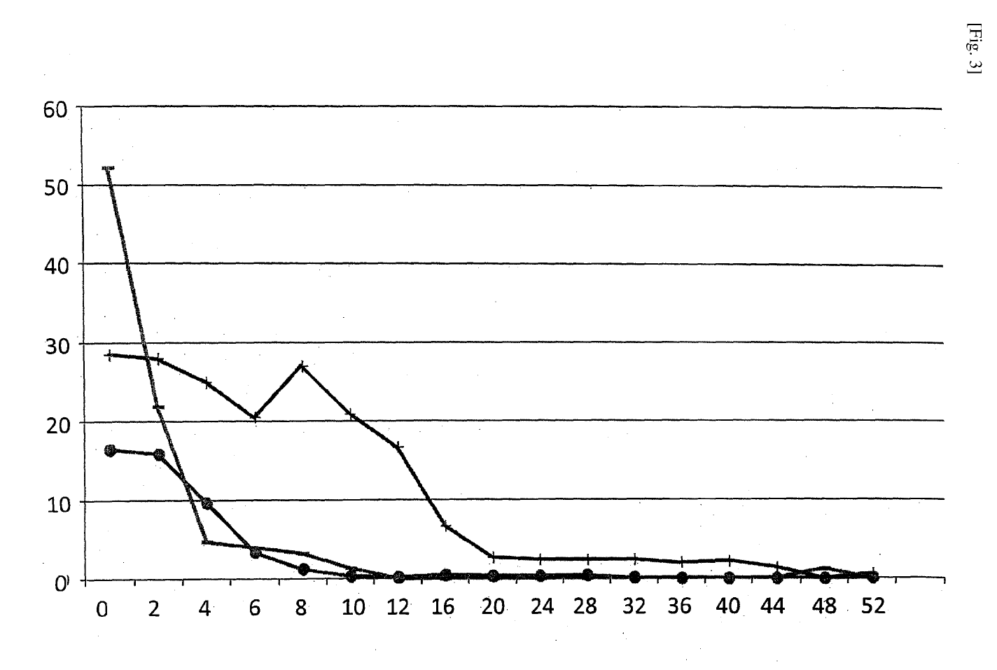

Three of the psoriatic erythroderma patients participated in the clinical trial of Example 1 had a history of anti-TNF-alpha antibody administration. These subjects were measured for PASI (Psoriasis Area and Severity Index) score, a measure of the degree of severity of psoriasis, over a time course. The results are presented in FIG. 3. PASI score was calculated by using the evaluation method given in Table 4.

TABLE 4
PASI Score Calculation Method

| | | | |
|---|---|---|---|
| Head | Trunk | Arms | Legs |

| | | |
|---|---|---|
| Observation | Erythema (a) | The skin condition of each subject was scored in 4 |
| of skin | Infiltration (b) | different parts of the body (head, trunk, Arms, and Legs) |
| | Desquamation | according to the following criteria. |
| | (c) | Absent: 0; mild: 1; moderate: 2; severe: 3; very severe: 4 |

| d = sum of a + b + c | | | | |
|---|---|---|---|---|
| Focus area (e) | The percentage area (%) of lesion in body surface area | | | |
| | was given focus area points, as follows: | | | |
| | 0%: 0; | | | |
| | less than 10%; 1; | | | |
| | 10% or more and less than 30%: 2; | | | |
| | 30% or more and less than 50%; 3; | | | |
| | 50% or more and less than 70%: 4; | | | |
| | 70% or more and less than 90%; 5; and | | | |
| | 90 to 100%: 6. | | | |
| d × e × coefficient by body | d × e × 0.1 | d × e × 0.3 | d × e × 0.2 | d × e × 0.4 |
| part | (I) | (II) | (III) | (IV) |

| | |
|---|---|
| PASI score | Sum of (I) + (II) + (III) + (IV) |

As described in FIG. 3, the PASI score decreased in each subject as the number of administration increased. The PASI score decreased to nearly 0 at the 10^{th}, the 12^{th}, and the 48^{th} week from the first administration in subjects F, H, and G, respectively. As demonstrated by these results, AM-14 was shown to have therapeutic effect for psoriatic erythroderma patients that had been administered with the anti-TNF-alpha antibodies.

Table 5 presents the histories of psoriasis therapeutic agent use for these three subjects.

TABLE 5
Subjects' Histories of Using Psoriasis Therapeutic Agent

| | Psoriasis | | |
|---|---|---|---|
| | therapeutic | | AM-14 |
| | agent | Reason for withdrawal of psoriasis | therapeutic |
| Subject | administered | therapeutic agent administered | effect |
| F | Adalimumab | Other | Present |
| | | (Change of treatment plan) | |
| G | Infliximab | Insufficient tolerability | Present |
| H | Adalimumab | Primary failure | Present |
| | Ustekinumab | Other (convulsive seizure) | |

Table 5 follows the same definitions used in Table 3. AM-14 was determined as being therapeutically effective when the PASI score at the 52^{th} week was lower than that measured before the administration of AM-14 in each patient. As can be seen in Table 5, AM-14 was shown to be therapeutically effective also in the patient (subject H) that did not respond to adalimumab. Although severe side effects occurred by administration of infliximab in the subject (subject G), and the subject G was a subject that was determined to be insufficient tolerability to infliximab, AM-14 was able to be continuously administered for 50 weeks, and shown therapeutic effect.

As demonstrated by these results, it was shown that, for a subject of psoriatic erythroderma that could not be treated with the anti-TNF-alpha antibody because the antiTNF-alpha antibody such as infliximab and adalimumab does not work, or causes severe side effects, AM-14 is able to be continuously administered over a long period of time and to improve psoriasis symptoms.

While the invention has been described in detail and with reference to specific embodiments thereof, it will be apparent to one skill in the art that various changes and modifications can be made therein without departing from the spirit and scope thereof.

This application is based on U.S. provisional patent application No. 62/041,862 filed on Aug. 26, 2014, the entire contents of which are incorporated hereinto by reference. All references cited herein are incorporated in their entirety.

### SEQUENCE LISTING FREE TEXT

SEQ ID NO: 1—Description of artificial sequence: amino acid sequence of AM-14_HCDR1

SEQ ID NO: 2—Description of artificial sequence: amino acid sequence of AM-14_HCDR2

SEQ ID NO: 3—Description of artificial sequence: amino acid sequence of AM-14_HCDR3

SEQ ID NO: 4—Description of artificial sequence: amino acid sequence of AM-14_LCDR1

SEQ ID NO: 5—Description of artificial sequence: amino acid sequence of AM-14_LCDR2

SEQ ID NO: 6—Description of artificial sequence: amino acid sequence of AM-14_LCDR3

SEQ ID NO: 7—Description of artificial sequence: amino acid sequence of AM-14_VH

SEQ ID NO: 8—Description of artificial sequence: amino acid sequence of AM-14_VL

1. A therapeutic agent for pustular psoriasis or psoriatic erythroderma that is administered to a psoriasis patient that has been administered with an anti-TNF-alpha antibody, comprising an IL-17RA antagonist as an active ingredient.

2. The therapeutic agent according to claim 1, wherein the anti-TNF-alpha antibody is at least one selected from adalimumab, infliximab, certolizumab pegol, certolizumab, and golimumab.

3. The therapeutic agent according to claim 1 or 2, wherein the IL-17RA antagonist is an anti-IL-17RA antibody or an antibody fragment thereof.

4. The therapeutic agent according to claim 3, wherein the anti-IL-17RA antibody is selected from the following a) and b):
a) a monoclonal antibody in which a complementarity determining region (hereinafter, referred to as CDR) 1, CDR2, and CDR3 of a heavy chain variable region (hereinafter, referred to as VH) of the antibody comprise the amino acid sequences shown in SEQ ID NOs:1, 2, and 3, respectively, and CDR1, CDR2, and CDR3 of a light chain variable region (hereinafter, referred to as VL) of the antibody comprise the amino acid sequences shown in SEQ ID NOs:4, 5, and 6, respectively; and
b) a monoclonal antibody in which VH of the antibody comprises the amino acid sequence shown in SEQ ID NO:7, and VL of the antibody comprises the amino acid sequence shown in SEQ ID NO:8.

5. The therapeutic agent according to any one of claims 1 to 4, wherein the clinical global impression (hereinafter, also referred to as CGI) of a patient after administration of the therapeutic agent becomes 2 or 1.

6. The therapeutic agent according to any one of claims 1 to 5, wherein the psoriasis area and severity index (hereinafter, referred to as PASI) score of a patient after administration of the therapeutic agent is lower than that before administration of the therapeutic agent.

7. A therapeutic agent for psoriasis that is administered to a psoriasis patient that cannot be treated with an anti-TNF-alpha antibody, comprising an IL-17RA antagonist as an active ingredient.

8. The therapeutic agent according to claim 7, wherein the patient that cannot be treated with the anti-TNF-alpha antibody is a patient that does not respond to the anti-TNF-alpha antibody or that is of insufficient tolerability to the anti-TNF-alpha antibody.

9. The therapeutic agent according to claim 7 or 8, the anti-TNF-alpha antibody is at least one selected from adalimumab, infliximab, certolizumab pegol, certolizumab, and golimumab.

10. The therapeutic agent according to any one of claims 7 to 9, wherein the IL-17RA antagonist is an anti-IL-17RA antibody or an antibody fragment thereof.

11. The therapeutic agent according to claim 10, wherein the anti-IL-17RA antibody is selected from the following a) and b):
a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and
b) a monoclonal antibody in which VH of the antibody comprises the amino acid sequence shown in SEQ ID NO:7, and VL of the antibody comprises the amino acid sequence shown in SEQ ID NO:8.

12. The therapeutic agent according to any one of claims 7 to 11, wherein the psoriasis is at least one selected from psoriasis vulgaris, psoriasis arthropica, pustular psoriasis, psoriatic erythroderma, and psoriasis guttata.

13. The therapeutic agent according to any one of claims 7 to 12, wherein the CGI of a patient after administration of the therapeutic agent becomes 2 or 1.

14. The therapeutic agent according to any one of claims 7 to 13, wherein the PASI score of a patient after administration of the therapeutic agent is lower than that before administration of the therapeutic agent.

15. A method for the treatment of pustular psoriasis or psoriatic erythroderma, comprising administering an IL-17RA antagonist to a psoriasis patient that has been administered with an anti-TNF-alpha antibody.

16. The method according to claim 15, wherein the anti-TNF-alpha antibody is at least one selected from adalimumab, infliximab, certolizumab pegol, certolizumab, and golimumab.

17. The method according to claim 15 or 16, wherein the IL-17RA antagonist is an anti-IL-17RA antibody or an antibody fragment thereof.

18. The method according to claim 17, wherein the anti-IL-17RA antibody is selected from the following a) and b):
a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and
b) a monoclonal antibody in which VH of the antibody comprises the amino acid sequence shown in SEQ ID NO:7, and VL of the antibody comprises the amino acid sequence shown in SEQ ID NO:8.

19. The method according to any one of claims 15 to 18, wherein the CGI of the patient after the administration of the IL-17RA antagonist becomes 2 or 1.

20. The method according to any one of claims 15 to 19, wherein the PASI score of a patient after the administration of the IL-17RA antagonist is lower than that before the administration of the antagonist.

21. The method according to any one of claims 15 to 20, wherein the IL-17RA antagonist is administered in a dose of 140 mg or more.

22. The method according to any one of claims 15 to 21, wherein the IL-17RA antagonist is administered in a dose of 140 mg or 210 mg.

23. The method according to any one of claims 15 to 22, wherein the IL-17RA antagonist is administered in a dose of 140 mg or 210 mg on the 1^{st} day, the 1^{st} week and the 2^{nd} week, and thereafter once every two weeks.

24. A method for the treatment of psoriasis, comprising administering an IL-17RA antagonist to a psoriasis patient that cannot be treated with an anti-TNF-alpha antibody.

25. The method according to claim 24, wherein the patient that cannot be treated with the anti-TNF-alpha antibody is a patient that is ineffective to the anti-TNF-alpha antibody or that is of insufficient tolerability to the anti-TNF-alpha antibody.

26. The method according to claim 24 or 25, wherein the anti-TNF-alpha antibody is at least one selected from adalimumab, infliximab, certolizumab pegol, certolizumab, and golimumab.

27. The method according to any one of claims 24 to 26, wherein the IL-17RA antagonist is an anti-IL-17RA antibody or an antibody fragment thereof.

28. The method according to claim 27, wherein the anti-IL-17RA antibody is selected from the following a) and b):
a) a monoclonal antibody in which CDR1, CDR2, and CDR3 of VH of the antibody comprise the amino acid sequences shown in SEQ ID NOs:1, 2, and 3, respectively, and CDR1, CDR2, and CDR3 of VL of the antibody comprise the amino acid sequences shown in SEQ ID NOs:4, 5, and 6, respectively; and
b) a monoclonal antibody in which VH of the antibody comprises the amino acid sequence shown in SEQ ID NO:7, and VL of the antibody comprises the amino acid sequence shown in SEQ ID NO:8.

29. The method according to any one of claims 24 to 28, wherein the psoriasis is at least one selected from psoriasis vulgaris, psoriasis arthropica, pustular psoriasis, psoriatic erythroderma, and psoriasis guttata.

30. The method according to any one of claims 24 to 29, wherein the CGI of a patient after the administration of the IL-17RA antagonist becomes 2 or 1.

31. The method according to any one of claims 24 to 30, wherein the PASI score of a patient after the administration of the IL-17RA antagonist is lower than that before the administration of the antagonist.

32. The method according to any one of claims 24 to 31, wherein the IL-17RA antagonist is administered in a dose of 140 mg or more.

33. The method according to any one of claims 24 to 32, wherein the IL-17RA antagonist is administered in a dose of 140 mg or 210 mg.

34. The method according to any one of claims 24 to 33, wherein the IL-17RA antagonist is administered in a dose of 140 mg or 210 mg on the 1^{st} day, the 1^{st} week and the 2^{nd} week, and thereafter once every two weeks.
